(19) United States
(12) Patent Application Publication
(10) Pub. No.: US 2023/0145200 A1
(43) Pub. Date: May 11, 2023

(54) COMPOSITIONS AND METHODS OF TREATING CANCER

(71) Applicant: Tillead Therapeutics, Inc., Mount Prospect, IL (US)

(72) Inventors: Hongyu Zhao, Libertyville, IL (US); Qian Chen, Chicago, IL (US); Guidong Zhu, Gurnee, IL (US)

(21) Appl. No.: 17/799,991

(22) PCT Filed: Feb. 18, 2021
(86) PCT No.: PCT/US21/18569

§ 371 Date: Aug. 16, 2022

Related U.S. Application Data
(60) Provisional application No. 62/983,238, filed on Feb. 28, 2020.

(51) Int. Cl. A61K 31/197; A61K 31/133; A61K 31/337; A61P 35/00; A61P 35/04; A61K 45/06; A61P 1/00; A61P 17/00

(52) U.S. Cl. CPC A61K 31/197; A61K 31/133; A61K 31/337; A61P 35/00; A61P 35/04; A61K 45/06; A61P 1/00; A61P 17/00

(57) ABSTRACT

The invention provides pharmaceutical compositions of caspase inhibitor(s), apoptosis inducer(s), and/or PD-1 pathway inhibitor(s), and methods for treating cancer and related diseases and conditions.

### PRIORITY CLAIMS AND RELATED APPLICATIONS

This application claims the benefit of priority to U.S. Provisional Application No. 62/983,238, filed Feb. 28, 2020, the entire content of which is incorporated herein by reference for all purposes.

### TECHNICAL FIELDS OF THE INVENTION

The invention generally relates to pharmaceuticals and therapeutic methods. More particularly, the invention provides novel pharmaceutical compositions of caspase inhibitors, apoptosis inducers, and/or PD-1 pathway inhibitors, and methods for treating cancer and related diseases and conditions.

### BACKGROUND OF THE INVENTION

Cancer is a group of diseases involving abnormal cell growth with the potential to invade or spread to other parts of the body. Cancer may be treated locally or systemically. Local treatment can sometime induce systemic efficacy, such as the abscopal effect in radiation therapy. However, abscopal effect is extremely rare and difficult to achieve (Seiwert T Y, J Clin Oncol. 2020; JCO2002046).

Apoptosis is an essential process in normal development, tissue homeostasis, and integrity of multicellular organisms. Damaged cells need to be removed during the process of metamorphosis, embryogenesis, pathogenesis, and tissue turnover (Jan R. Adv Pharm Bull. 2019, 9:205). Apoptosis is involved in the oncogenesis and tumor repopulation (Huang Q, et al. Nat Med 2011; 17:860-6) and immune escape (Han C, et al. Nat Immunol. 2020; 21:546-54).

Caspase system is considered the executioner of apoptosis by degrading cellular components required for normal cellular functions such as cytoskeletal and nuclear proteins. The caspases belong to a class of structurally related cysteine proteases that contains the plant metacaspases, mammalian and plant proteases of the legumain family, the eukaryotic protease separase, and several bacterial proteases. Caspases display very narrow preferences at the P1 position, primarily recognizing aspartic acid, but also cleave substrates after glutamic acid and phosphoserine residues (Kasperkiewicz P, et al. FEBS J. 2017; 284:1518-39). In response to apoptosis stimuli, such as growth factor deprivation, cytoskeletal disruption, oxidative stress, DNA damage and accumulation of unfolded proteins, initiator caspases (caspase-2, -8, -9, or -10) are activated to cleave and activate the zymogenic forms of “executioner” caspases (e.g. caspase-3 or -7), resulting in the proteolytic cleavage of specific cellular substrates and, consequently, cell death (Duckett C S. et al. Mol Cell Biol. 1998; 18:608-15).

While evading apoptosis signals is considered a hallmark of cancer (Hanahan D, Weinberg R A. Cell 2011; 144:646-74), caspase mutations are rare in cancer, suggesting an essential role in tumor homeostasis (Boudreau M W, et al. ACS Chem Biol. 2019; 14:2335-48). Activated caspases are necessary for apoptosis to occur, but they are dispensable for cell death and the apoptotic clearance of cells in vivo. Paradoxically, apoptosis can also cause unwanted effects that may even promote cancer through apoptosis induced proliferation (Ichim G, et al. Nat Rev Cancer. 2016; 16:539). Caspases can promote proliferation through autonomous regulation of the cell cycle, as well as by induction of secreted signals, which have a profound impact in neighboring tissues (Pérez-Garijo A, et al. Seminars in cell & developmental biology 2018; 82:86-95. Academic Press). Genetic knockout of caspases in tumor reduces tumor fitness and tumor repopulation (Zhao M, et al. Aging 2020; 12:21758).

Billions of cells undergo apoptosis per day in a human body, during which DNA is released into cytosol. It is important to keep the process immunologically silent for the survival of the host. Apoptotic cells actively communicate with their environment to suppress anti-inflammatory immune response and instruct specific subsets of phagocytes to clear dying cellular debris (Fogarty C E, et al. Curr Topics Dev Biol 2015; 114:241-265. Academic Press).

Caspases play a role in evading the immune response of apoptotic cells. The release of genomic DNA and mtDNA (mitochondria DNA) in stressed cells triggers cyclic GMP-AMP Synthase-Stimulator of Interferon Genes (cGAS-STING) signaling, IRF3 activation, and type I interferon (IFN) production. However, the concurrent release of cytochrome c, formation of the apoptosome, and activation of the downstream effectors caspase-3 and caspase-7 mitigates this immune activation signal, resulting in a silent apoptotic cell death (Chen Y, et al. Front Physiol. 2018; 18:1487).

In the absence of caspase activation, however, a profound protection from both DNA and RNA virus infection was observed (Rongvaux A, et al. Cell 2014; 159:1563-77). Caspases were also found to prevent the induction and secretion of the anti-viral factor IFNβ during the replicative infection of Kaposi's sarcoma-associated herpesvirus (KSHV), the causative agent of AIDS-defining tumor Kaposi's sarcoma (KS). The reduced IFNβ production allows for high viral gene expression and viral replication (Tabtieng T, et al. J Virol. 2018; 92:e00078-18). Caspases were also found to involve in regulating both DNA and RNA virus-triggered host defense by cleaving the key STING pathway components such as cGAS, MAVS, and IRF3 to prevent cytokine overproduction. Deficiency in apoptotic caspases was linked to elevated IFN production during virus infection (Ning X, et al. Mole Cell. 2019; 74:19-31).

Although chemical agents that promote apoptosis such as Venetoclax have been approved by the FDA as cancer therapies in some blood cancers, apoptosis blockers may induce tumor control through various non-apoptotic mechanisms (so-called caspase-independent cell deaths, CICD). Intratumoral administration significantly increases tumor drug exposure to achieve chemical ablation with reduced systemic side effects. More importantly, local injection of caspase inhibitors that prime immune response may induce the abscopal effect and functions as an in situ vaccine.

Immune checkpoint inhibitors (ICIs) such as PD-1 (programmed cell death protein 1) antagonists, PD-L1 (programmed cell death ligand 1) antagonists, and CTLA4 antagonists have produced durable and deep response in several metastatic cancers and significantly prolonged the survival of the patients treated. However, the overall respond rate is low for these therapies. In several major cancers such as breast, pancreatic, and prostate cancers the respond rate of ICIs as last line therapy is negligible (Arnaud-Coffin P. et al. Intl. J. Cancer 2019; 145:639-648). Local immunotherapies in combination of systemic ICI may result in improved cancer treatment.

The therapeutics and methods currently available for treating cancer are inadequate. There remains an urgent and ongoing need for novel and improved therapeutics to effectively treat cancers and related diseases and conditions.

### SUMMARY OF THE INVENTION

The invention is based, in part, on the unexpected discovery novel pharmaceutical compositions of caspase inhibitors, apoptosis inducers, and/or PD-1 pathway inhibitors, and methods thereof for treating cancer and related diseases and conditions.

In one aspect, the invention generally relates to a method for treating cancer, comprising intratumorally administering, simultaneously or sequentially, to a subject in need thereof a therapeutically effective amount of a caspase inhibitor and a therapeutically effective amount of an apoptosis inducer. In certain embodiments, the method further comprises administering to the subject a therapeutically effective amount of a PD-1 pathway inhibitor.

In another aspect, the invention generally relates to a method for treating cancer, comprising intratumorally administering to a subject in need thereof a therapeutically effective amount of emricasan, or a pharmaceutically acceptable form thereof.

In yet another aspect, the invention generally relates to a method for treating cancer, comprising subcutaneously administering to a subject in need thereof a therapeutically effective amount of emricasan, or a pharmaceutically acceptable form thereof, and intravenously administering to the subject a therapeutically effective amount of docetaxel, or a pharmaceutically acceptable form thereof.

In yet another aspect, the invention generally relates to a pharmaceutical composition comprising emricasan, or a pharmaceutically acceptable form thereof, and docetaxel, or a pharmaceutically acceptable form thereof, and one or more pharmaceutically acceptable excipients, carriers, or diluents.

In yet another aspect, the invention generally relates to a unit dosage form comprising a pharmaceutical composition disclosed herein.

In yet another aspect, the invention generally relates to use of emricasan, or a pharmaceutically acceptable form thereof, for the manufacture of a medicament for the treatment of cancer, or a related disease or condition.

In yet another aspect, the invention generally relates to use of emricasan, or a pharmaceutically acceptable form thereof, and docetaxel, or a pharmaceutically acceptable form thereof, for the manufacture of a medicament for the treatment of cancer, or a related disease or condition.

In yet another aspect, the invention generally relates to use of emricasan, or a pharmaceutically acceptable form thereof, for treating cancer, or a related disease or condition.

In yet another aspect, the invention generally relates to a solid form of emricasan CH₃N(CH₂CH₂OH)₂ salt.

### BRIEF DESCRIPTION OF THE DRAWINGS

FIG. 1 shows exemplary data of emricasan blocking apoptosis in MC38 cells.

FIG. 2 shows exemplary data of emricasan blocking apoptosis induced by simvastatin in MC38 cells.

FIG. 3 shows exemplary data of emricasan blocking apoptosis induced by venetoclax in MC38 cells.

FIG. 4 shows exemplary data of emricasan blocking apoptosis induced by doxorubicin in MC38 cells.

FIG. 5 shows exemplary data of emricasan blocking apoptosis induced by docetaxel in MC38 cells.

FIG. 6 shows exemplary data of emricasan slightly enhancing IFNβ production in MC38 cells.

FIG. 7 shows exemplary data of emricasan significantly enhancing IFNβ production in simvastatin treated MC38 cells.

FIG. 8 shows exemplary data of emricasan significantly enhancing IFNβ production in doxorubicin treated MC38 cells.

FIG. 9 shows exemplary data of emricasan significantly enhancing IFNβ production in docetaxel treated MC38 cells.

FIG. 10a (MC38), 10b (A549), 10c (MiaPaca-2) show exemplary data on docetaxel activating caspase more efficiently at ˜1 μM and 10 μM of emricasan fully inhibiting the caspase activity in tumor cells.

FIG. 11a (MC38), 11b (A549), 11c (MiaPaca-2) show exemplary data on docetaxel killing tumor cells more efficiently at ˜1 μM with 10 μM of emricasan.

FIG. 12 shows exemplary data of in vivo efficacy of emricasan amine salt in the treatment of injection site tumor given intratumorally of 10 mpk (q.d) for 4 days in the MC38 model.

FIG. 13 shows exemplary data of in vivo efficacy of emricasan amine salt in the treatment of distal tumor given intratumorally at 10 mpk (q.d) for 4 days in the MC38 model.

FIG. 14 shows exemplary data of in vivo efficacy of combination of docetaxel (intraperitoneal, 30 mpk, single dose) and emricasan (intratumoral, 10 mpk, q.d for 3 days after docetaxel administration) in the MC38 model.

FIG. 15 shows exemplary data of in vivo efficacy of combination of docetaxel (intravenous, 30 mpk single dose) and emricasan (intratumoral, 10 mpk q.d for 4 days after docetaxel administration) in the MC38 model.

FIG. 16 shows exemplary data of in vivo efficacy for the rechallenged tumor in the cured mice from the studies shown in FIG. 15 (5 mice from the emricasan group, E1, E2, E3, E4, E5; and 1 mouse from the combo group, C1) in the MC38 model.

FIG. 17 shows exemplary data of in vivo efficacy of combination of docetaxel (1 mpk, 1D; 5 mpk, 5D, q.d) and emricasan (10 mpk, 10E, q.d) co-dosed intratumorally for 4 days in the MC38 model.

FIG. 18 shows exemplary data of in vivo efficacy of combination of docetaxel (intravenous, 50 mpk, single dose) and emricasan (intraperitoneal, 20 mpk, b.i.d for 3 days after docetaxel administration) in the MC38 model.

FIG. 19 shows exemplary data of in vivo efficacy of emricasan amine salt given intratumorally at 5 mpk and 10 mpk (q.d) for 4 days in the B16 model.

FIG. 20 shows exemplary data of in vivo efficacy of emricasan amine salt given intratumorally at 5 mpk and 10 mpk (q.d) for 4 days in the CT26 model.

FIG. 21 shows exemplary data of docetaxel (1 μM) and its combination with emricasan (10 μM) upregulating PD-L1 expression in MC38 cells.

### DEFINITIONS

Certain technical and scientific terms are specifically defined below. Unless otherwise defined, all technical and scientific terms used herein have the same meaning as commonly understood by one of ordinary skill in the art to which this disclosure relates. General principles of organic chemistry, as well as specific functional moieties and reactivity, are described in “Organic Chemistry”, Thomas Sorrell, University Science Books, Sausalito: 2006. In case of conflict, the present specification, including definitions, will control.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Unless otherwise required by context, singular terms shall include pluralities and plural terms shall include the singular.

Any example(s) following the term “e.g.” or “for example” is not meant to be exhaustive or limiting.

The terms “a,” “an,” and “the,” include their corresponding plural references unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, “at least” a specific value is understood to be that value and all values greater than that value. The terms “at least one” item or “one or more” item each include a single item selected from the list as well as mixtures of two or more items selected from the list.

As used herein, the terms “administration” and “administering” of a disclosed compound refer to the delivery to a subject of a compound as described herein, or a prodrug or other pharmaceutically acceptable form thereof, using any suitable formulation or route of administration, as discussed herein. The term “administered individually” as used herein in relation to the administration of medicaments refers to the administration of individual medicaments (via the same or an alternative route) at different times. The term “administered simultaneously” as used herein in relation to the administration of medicaments refers to the administration of medicaments such that the individual medicaments are present within a subject at the same time. The term “systemically administered” means a drug is given orally or parenterally.

As used herein, the term “caspase inhibitor” refers any chemical compound or biological molecule that binds and inhibits the activity of caspases.

As used herein, the term “immune response” refers to any one or more of the following: specific immune response, non-specific immune response, both specific and non-specific response, innate response, primary immune response, adaptive immunity, secondary immune response, memory immune response, immune cell activation, immune cell-proliferation, immune cell differentiation, and cytokine expression.

As used herein, the term “in combination” refers to the use of more than one therapies (e.g., a caspase inhibitor and other agents). The use of the term “in combination” does not restrict the order in which therapies (e.g., a caspase inhibitor and other agents) are administered to a subject with a disorder. A first therapy (e.g., an agent that initiates apoptosis and other agents) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of other therapy (e.g., a caspase inhibitor and other agents) to a subject with a disorder.

As used herein, the term “intratumorally administered” means that a solution or suspension is injected (e.g., through a needle) directly into the tumor lesion or mass.

As used herein, the term “PD-1 antagonist” or “PD-1 pathway antagonist” refers to any chemical compound or biological molecule that blocks binding of PD-L1 expressed on a cancer cell to PD-1 expressed on an immune cell (e.g., T-cell, B-cell, or NK-cell).

As used herein, a “pharmaceutically acceptable form” of a disclosed compound includes, but is not limited to, pharmaceutically acceptable salts, esters, hydrates, solvates, isomers, prodrugs, and isotopically labeled derivatives of disclosed compounds. In one embodiment, a “pharmaceutically acceptable form” includes, but is not limited to, pharmaceutically acceptable salts, esters, isomers, prodrugs and isotopically labeled derivatives of disclosed compounds. In some embodiments, a “pharmaceutically acceptable form” includes, but is not limited to, pharmaceutically acceptable salts, esters, stereoisomers, prodrugs and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)⁴ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a pharmaceutically acceptable ester. As used herein, the term “pharmaceutically acceptable ester” refers to esters that hydrolyze in vivo and include those that break down readily in the human body to leave the parent compound or a salt thereof. Such esters can act as a prodrug as defined herein. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfinic acids, sulfonic acids and boronic acids. Examples of esters include formates, acetates, propionates, butyrates, acrylates and ethylsuccinates. The esters can be formed with a hydroxy or carboxylic acid group of the parent compound.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable” excipient, carrier, or diluent refers to any inactive substance that is suitable for use in a formulation for the delivery of a therapeutic agent. Each carrier must be “acceptable” in the sense of being compatible with the other ingredients of the formulation and not injurious to the patient. A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like), dextrose, vegetable oils (such as olive oil), saline, buffer, buffered saline, and isotonic agents such as sugars, poly alcohols, sorbitol, and sodium chloride.

As used herein, the term “radiation therapy”, also called radiation oncology, radiation therapy, or therapeutic radiology refers to the use of ionizing radiation to destroy cancer cells.

As used herein, the term “subject” (alternatively “patient”) as used herein refers to a mammal that has been the object of treatment, observation, or experiment. The mammal may be male or female. The mammal may be one or more selected from the group consisting of humans, non-human primates, bovine (e.g., cows), porcine (e.g., pigs), ovine (e.g., sheep), capra (e.g., goats), equine (e.g., horses), canine (e.g., domestic dogs), feline (e.g., house cats), Lagomorpha (rabbits), rodents (e.g., rats or mice), Procyon lotor (e.g., raccoons). In particular embodiments, the subject is human.

As used herein, the term “therapeutically effective amount” refers to the dose of a therapeutic agent or agents sufficient to achieve the intended therapeutic effect with minimal or no undesirable side effects. A therapeutically effective amount can be determined by a skilled physician, e.g., by first administering a low dose of the pharmacological agent(s) and then incrementally increasing the dose until the desired therapeutic effect is achieved with minimal or no undesirable side effects.

As used herein, the terms “treatment” of or “treating” a disease or disorder refer to a method of reducing, delaying or ameliorating such a condition before or after it has occurred. Treatment may be directed at one or more effects or symptoms of a disease and/or the underlying pathology. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

In particular, “treatment” of or “treating” cancer refers to achieving at least one positive therapeutic effect, such as for example, reduced number of cancer cells, reduced tumor size, reduced rate of cancer cell infiltration into peripheral organs, or reduced rate of tumor metastasis or tumor growth. Such “treatment” may result in a slowing, interrupting, arresting, controlling, or stopping of the progression of a cell-proliferation disorder as described herein but does not necessarily indicate a total elimination of the cell-proliferation disorder or the symptoms of the cell-proliferation disorder. Positive therapeutic effects achieved may be any of PR (partial response), CR (complete response), OR (overall response), PFS (progression free survival), DFS (disease free survival), and OS (overall survival). PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to I or untreated individuals or patients.

As used herein, the term “tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors.

### DETAILED DESCRIPTION OF THE INVENTION

The invention provides novel pharmaceutical compositions of caspase inhibitors, apoptosis inducers, and/or PD-1 pathway inhibitors, and methods thereof for treating cancer and related diseases and conditions. In particular, the invention provides a novel approach to cancer treatment based on the discovery that activated caspases play a key role in stimulating tumor growth and evading local and systemic immune surveillance during induced and spontaneous apoptosis.

Caspases belong to a class of structurally related cysteine proteases that degrade cellular components required for normal cellular functions such as cytoskeletal and nuclear proteins during apoptosis. The activity of caspases can be inhibited by chemical compounds that bind to the proteins in either orthosteric or allosteric sites. The inhibitors can be either reversible or irreversible. The irreversible inhibitors bind to the caspase protein and form a covalent bond with a cysteine residue and blocks the binding of the endogenous substrates and their degradation.

Caspase activation is an integral step in cell apoptosis and other fundamental processes. While low level caspase activity promotes tumorigenesis, tumor repopulation, and immune escape, high level caspase activity leads to apoptosis. Inhibition of caspases after spontaneous or induced apoptosis by an apoptosis initiating agent can control tumor by blocking tumor repopulation and enhancing innate immunity via type I interferon production and other inflammatory effects such as NF-KB activation. Intratumorally administration of caspase inhibitors significantly increases tumor drug concentration and reduces systemic side effects. Addition of PD-1 pathway antagonists further enhances local and systemic control of tumor growth. The invention provides methods of treating cancer using caspase inhibitors and combination thereof with apoptosis initiating agents and PD-1 pathway antagonists.

In one aspect, the invention generally relates to a method for treating cancer, comprising intratumorally administering, simultaneously or sequentially, to a subject in need thereof a therapeutically effective amount of a caspase inhibitor and a therapeutically effective amount of an apoptosis inducer.

In certain embodiments, the method further comprises administering to the subject a therapeutically effective amount of a PD-1 pathway inhibitor.

In certain embodiments, the caspase inhibitor inhibits the activity of at least one caspase chosen from caspase-2, caspase-3, caspase-6, caspase-7, caspase-8, caspase-9 and caspase-10.

Exemplary caspase inhibitors include Cbz-VAD-FMK (ZVAD), Ac-YVAD-CHO, pralnacasan (VX-740), belnacasan (VX-765), VX-043198, emricasan (IDN-6556), nivocasan (GS-9450) and NCX-1000.

In certain embodiments, the caspase inhibitor is emricasan (IDN-6556), (S)-3-((S)-2-(2-((2-(tert-butyl)phenyl)amino)-2-oxoacetamido)propanamido)-4-oxo-5-(2,3,5,6-tetrafluorophenoxy)pentanoic acid), represented by Formula I below, or a pharmaceutically acceptable form thereof.

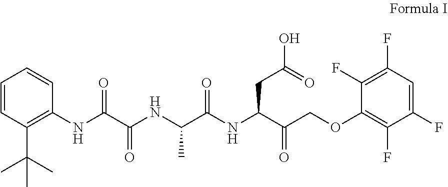 [SMILES: C[C@H](NC(=O)C(=O)Nc1ccccc1C(C)(C)C)C(=O)N[C@@H](CC(=O)O)C(=O)COc1c(F)c(F)cc(F)c1F]

In certain embodiments, emricasan is in the form of a base addition salt. In certain embodiments, the base addition salt is formed by emricasan and CH₃N(CH₂CH₂OH)₂ (N-methyl diethanolamine).

Furthermore, the activity of caspases can be inhibited by linking a caspase inhibitor to an E3 ligase through a linker to form a proteolysis targeting chimera (PROTAC). In certain embodiments, the caspase inhibitor is chosen from the group consisting of PROTACs made from an E3 ligase, a chemical linker, and a molecule chosen from the group consisting of Cbz-VAD-FMK (ZVAD), Ac-YVAD-CHO, pralnacasan (VX-740), belnacasan (VX-765), VX-043198, emricasan, nivocasan (GS-9450), NCX-1000, and pharmaceutically acceptable form thereof.

Apoptosis can be initiated by different types of cellular stress, including but not limited to oxidative stress, radiation, physical trauma, chemotherapeutic drugs, infectious agents including viruses and bacterial toxins, genomic DNA, and mtDNA.

In certain embodiments, the apoptosis is initiated by microtubule-stabilizing agents. In one embodiment, the apoptosis is initiated by a taxane family of chemotherapeutics. Taxanes stabilize tubulin polymerization, resulting in arrest at the G2/M phase of the cell cycle and apoptotic cell death. In another embodiment, the apoptosis is initiated by a taxane chosen from paclitaxel, docetaxel, cabazitaxel, or protein-bound paclitaxel. In another embodiment, the apoptosis is initiated by docetaxel.

In certain embodiments, the apoptosis inducer is docetaxel, or a pharmaceutically acceptable form thereof.

In certain embodiments, the apoptosis is initiated by radiation therapy. Radiation therapy damages DNA and permeabilizes mitochondria, resulting in the release of DNA into cytosol and initiation of apoptosis. In one embodiment, the radiation therapy uses ionizing radiation generated by x-rays or gamma rays. In another embodiment, the radiation therapy uses ionizing radiation generated by electrons, protons, neutrons, carbon ions, alpha particles, and beta particles.

In certain embodiments, the apoptosis is initiated by 3-hydroxy-3-methyl-glutaryl-CoA (HMGCoA) reductase inhibitors (statins), and pharmaceutically acceptable forms thereof. Statins induce apoptosis by decreasing the mitochondrial transmembrane potential, increasing the activation of caspase-9 and caspase-3, enhancing Bim expression, and inducing cell-cycle arrest at G1 phase through inhibition of Ras/extracellular signal-regulated kinase and Ras/mammalian target of rapamycin pathways.

Exemplary HMGCoA reductase inhibitors include atorvastatin, fluvastatin, lovastatin, pravastatin, rosuvastatin, simvastatin, pitavastatin, and pharmaceutically acceptable forms thereof. In another embodiment, the HMGCoA reductase inhibitor is simvastatin, and pharmaceutically acceptable forms thereof.

In certain embodiments, the apoptosis is initiated by a Bcl inhibitor. Bcl family of proteins are known anti-apoptotic proteins and their inhibitors are known to initiate apoptosis processes. Exemplary Bcl inhibitors include APG-2575, navitoclax (ABT-263), ABT-737, venetoclax, and pharmaceutically acceptable forms thereof. In certain embodiments, the Bcl inhibitor is venetoclax. Other direct and indirect Bcl inhibitors include gossypol, epigallocatechin gallate, licochalcone A, HA14-1, TW-37, EM20-25.

In certain embodiments, the apoptosis is initiated by DNA damaging agents. In one embodiment, the apoptosis is initiated by an anthracycline family of chemotherapeutics. Anthracyclines are known to initiate apoptosis and activate caspases by intercalating DNA. In another embodiment, the apoptosis is initiated by an anthracycline chosen from doxorubicin, daunorubicin, epirubicin, idarubicin, mitoxantrone, valrubicin, and pharmaceutically acceptable forms thereof. In another embodiment, the apoptosis is initiated by doxorubicin, and pharmaceutically acceptable forms thereof.

Several other chemotherapeutic agents, including but not limited to tyrosine kinase inhibitors, cytotoxic agents, alkylating agents, angiogenesis inhibitors, proteasome inhibitors, anti-metabolites, growth factor receptor antagonists, reactive oxygen species generators, also initiate apoptosis. In another aspect, the apoptosis is initiated by a chemotherapeutic agent that is chosen from AEE788, altretamine, AMG510, AMG706, aminopterin, anthracenedione, ARQ197, nelarabine, asparaginase, axitinib, azacitidine, AZD0530, AZD2171, AZD6244, belotecan, bendamustine, beta-lapachone, bevacizumab, BI2536, BIBF1120, bleomycin, BMS-275183, bortezomib, bosutinib, busulfan, camptothecin, capecitabine, carboplatin, carboquone, carmustine, CEP701, cetuximab, chlorambucil, chlormethine, CI-1033, cisplatin, cladribine, clofarabine, cyclophosphamide, cytarabine, dacarbazine, dactinomycin, dasatinib, docetaxel, EKB-569, EMD-72000, enocitabine, enzastaurin, epirubicin, erlotinib, ET-743, etoposide, everolimus, EXEL0999, EXEL7647, floxuridine, fludarabine phosphate, fotemustine, gefitinib, gemcitabine, GX15-070, HKI-272, hydroxyurea, ICR-62, idarubicin, ifosfamide, imatinib, irinotecan, ispinesib, ixabepilone, lapatinib, larotaxel, leucovorin, lomustine, lovastatin, mannosulfan, mechlorethamine, melphalan, mercaptopurine, methotrexate, mitomycin C, mitoxantrone, MLN-518, MS-275, napabucasin, nedaplatin, nilotinib, nimustine, ortataxel, osimertinib, oxaliplatin, paclitaxel, panitumumab, pazopanib, PD0325901, pemetrexed, pentostatin, PKC-412, plicamycin, prednimustine, procarbazine, PTK787, raltitrexed, ranimustine, rubitecan, sapacitabine, satraplatin, seliciclib, semustine, sorafenib, streptozocin, sunitinib, temozolomide, temsirolimus, teniposide, tesetaxel, thioguanine, thiotepa, topotecan, treosulfan, triaziquone, triethylenemelamine, triplatin tetranitrate, trofosfamide, uracil mustard, uramustine, vandetanib, vinblastine, vincristine, vindesine, vinorelbine, vinzolidine, XL119, XL880, and ZD6474.

PD-1 pathway is a major immune inhibitory pathway and PD-1 antagonists are known to promote immune response and are used to treat cancer in medical practices.“PD-1 antagonist” or “PD-1 pathway antagonist” refers to any chemical compound or biological molecule that blocks binding of PD-L1 expressed on a cancer or an immune cell to PD-1 expressed on an immune cell (T-cell, B-cell, or NK-cell). Alternative names or synonyms for PD-1 and its ligands include: PDCD1, PD1, CD279, and SLEB2 for PD-1; PDCD1L1, PDL1, B7H1, B7-4, CD274, and B7-H for PD-Ll. In any of the treatment methods, medicaments and uses disclosed in which a human individual is being treated, the PD-1 antagonist blocks binding of human PD-Ll to human PD-1, and preferably blocks binding of both human PD-Ll and PD-L2 to human PD-1.

Exemplary PD-1 pathway antagonists include pembrolizumab, nivolumab, atezolizumab, durvalumab, avelumab, cemiplimab, and pharmaceutically acceptable forms thereof. Other PD-1 pathway antagonists include but not limited to spartalizumab (PDR001), camrelizumab (SHR1210), sintilimab (IBI308), tislelizumab (BGB-A317), toripalimab (JS 001), dostarlimab, INCMGA00012, AMP-224, AMP-514, KN035, CK-301, AUNP12, CA-170, BMS-986189.

Cancer being treated by the methods of the invention may be localized or metastatic. In certain embodiments, the cancer is localized. In certain embodiments, the cancer is metastatic.

In certain embodiments, the cancer is selected from breast cancer, small cell lung cancer, non-small cell lung cancer, prostate cancer, gastric cancer, renal cell carcinoma, ovarian cancer, cervical cancer, colorectal cancer, hepatocellular carcinoma, glioblastoma, melanoma, basal cell carcinoma, cutaneous squamous cell carcinoma, Bowen's disease, pancreatic ductal carcinoma, head and neck squamous cell carcinoma, lip squamous cell carcinoma, buccal mucosa squamous cell carcinoma, oral tongue squamous cell carcinoma, oral squamous cell carcinoma, salivary mucoepidermoid carcinoma, and endometrial carcinoma.

In certain embodiments, the cancer is selected from breast cancer, non-small cell lung cancer, prostate cancer, head and neck squamous cell carcinoma, renal cell carcinoma, hepatocellular carcinoma, and melanoma.

In another aspect, the invention generally relates to a method for treating cancer, comprising intratumorally administering to a subject in need thereof a therapeutically effective amount of emricasan, or a pharmaceutically acceptable form thereof.

In certain embodiments, the method further comprises intratumorally administering to the subject a therapeutically effective amount of docetaxel, or a pharmaceutically acceptable form thereof.

In certain embodiments, the therapeutically effective amount of emricasan and the therapeutically effective amount of docetaxel are co-administered as a single intratumoral administration.

In certain embodiments, the therapeutically effective amount of emricasan and the therapeutically effective amount of docetaxel are co-administered as separate intratumoral administrations.

In certain embodiments, the weight ratio of docetaxel and emricasan is in the range of about 1:20 to about 1:2 (e.g., about 1:20 to about 1:5, about 1:20 to about 1:7, about 1:20 to about 1:10, about 1:15 to about 1:2, about 1:10 to about 1:2, about 1:7 to about 1:2).

In certain embodiments, the method further comprises locally administering to the subject a therapeutically effective amount of radiation therapy.

In certain embodiments, cancer growth at a site away from the site of intratumoral administration is suppressed.

In certain embodiments, the intratumoral administration results in systemic suppression of cancer growth.

In certain embodiments, the method further comprises administering to the subject a PD-1 pathway inhibitor.

In certain embodiments, the cancer is localized. In certain embodiments, the cancer is metastatic.

In certain embodiments, the cancer is selected from breast cancer, small cell lung cancer, non-small cell lung cancer, prostate cancer, gastric cancer, renal cell carcinoma, ovarian cancer, cervical cancer, colorectal cancer, hepatocellular carcinoma, glioblastoma, melanoma, basal cell carcinoma, cutaneous squamous cell carcinoma, Bowen's disease, pancreatic ductal carcinoma, head and neck squamous cell carcinoma, lip squamous cell carcinoma, buccal mucosa squamous cell carcinoma, oral tongue squamous cell carcinoma, oral squamous cell carcinoma, salivary mucoepidermoid carcinoma, and endometrial carcinoma.

In certain embodiments, the cancer is selected from breast cancer, non-small cell lung cancer, prostate cancer, head and neck squamous cell carcinoma, renal cell carcinoma, hepatocellular carcinoma, and melanoma.

In certain embodiments, emricasan is administered at a daily dosage in the range of about 0.5 mg to about 100 mg (e.g., about 1 mg to about 100 mg, about 5 mg to about 100 mg, about 10 mg to about 100 mg, about 25 mg to about 100 mg, about 0.5 mg to about 50 mg, about 0.5 mg to about 25 mg, about 0.5 mg to about 10 mg, about 0.5 mg to about 2 mg) for a time period of about 1 to about 10 days (e.g., about 1 to about 7 days, about 7 to about 10 days).

In certain embodiments, docetaxel is administered at a daily dosage in the range of about 0.5 mg to about 50 mg (e.g., about 1 mg to about 50 mg, about 5 mg to about 50 mg, about 10 mg to about 50 mg, about 25 mg to about 50 mg, about 0.5 mg to about 25 mg, about 0.5 mg to about 10 mg, about 0.5 mg to about 2 mg) for a time period of about 1 to about 10 days (e.g., about 1 to about 7 days, about 7 to about 10 days).

In yet another aspect, the invention generally relates to a method for treating cancer, comprising subcutaneously administering to a subject in need thereof a therapeutically effective amount of emricasan, or a pharmaceutically acceptable form thereof, and intravenously administering to the subject a therapeutically effective amount of docetaxel, or a pharmaceutically acceptable form thereof.

In yet another aspect, the invention generally relates to a pharmaceutical composition comprising emricasan, or a pharmaceutically acceptable form thereof, and docetaxel, or a pharmaceutically acceptable form thereof, and one or more pharmaceutically acceptable excipients, carriers, or diluents.

In certain embodiments, the pharmaceutical composition is an aqueous solution.

In certain embodiments, the pharmaceutical composition comprises a mixture of TWEEN80 and PEG300 and/or a mixture of TWEEN80 and ethanol, which can be diluted to an aqueous solution prior to injection.

In certain embodiments, the pharmaceutical composition is characterized by a concentration of emricasan in the range of about 1 mg/mL to about 40 mg/mL (e.g., about 5 mg/mL to about 40 mg/mL, about 10 mg/mL to about 40 mg/mL, about 1 mg/mL to about 25 mg/mL, about 1 mg/mL to about 10 mg/mL), a concentration of docetaxel in the range of about 1 mg/mL to about 20 mg/mL (e.g., about 5 mg/mL to about 20 mg/mL, about 10 mg/mL to about 20 mg/mL, about 1 mg/mL to about 10 mg/mL, about 1 mg/mL to about 5 mg/mL), and a pH in the range of about 5.0 to about 7.0 (e.g., about 5.5 to about 7.0, about 5.0 to about 6.5, about 5.5 to about 6.5).

In certain embodiments, the pharmaceutical composition of the invention is stable at −20° C. conditions for at least 6 months.

In yet another aspect, the invention generally relates to a unit dosage form comprising a pharmaceutical composition disclosed herein.

In yet another aspect, the invention generally relates to use of emricasan, or a pharmaceutically acceptable form thereof, for the manufacture of a medicament for the treatment of cancer, or a related disease or condition.

In yet another aspect, the invention generally relates to use of emricasan, or a pharmaceutically acceptable form thereof, and docetaxel, or a pharmaceutically acceptable form thereof, for the manufacture of a medicament for the treatment of cancer, or a related disease or condition.

In yet another aspect, the invention generally relates to use of emricasan, or a pharmaceutically acceptable form thereof, for treating cancer, or a related disease or condition.

In certain embodiments, the use of emricasan, or a pharmaceutically acceptable form thereof, is in combination with use of docetaxel, or a pharmaceutically acceptable form thereof, for treating cancer, or a related disease or condition. In certain embodiments, such use is in further combination with use of a PD-1 pathway inhibitor for treating cancer, or a related disease or condition.

In yet another aspect, the invention generally relates to a solid form of emricasan CH₃N(CH₂CH₂OH)₂ salt.

In certain embodiments, the solid form is substantially pure. In certain embodiments, the solid form is substantially crystalline.

In certain embodiments, the invention provides a method of treating cancer by enhancing innate immune responses in a subject in need thereof comprising the steps of: (1) administering to the subject a therapeutically effective amount of an apoptosis initiating agent chosen from the group consisting of doxorubicin, daunorubicin, epirubicin, idarubicin, mitoxantrone, valrubicin, and pharmaceutically acceptable forms thereof; and (2) administering to the subject a therapeutically effective amount of caspase inhibitor chosen from the group consisting of Cbz-VAD-FMK (ZVAD), Ac-YVAD-CHO, pralnacasan (VX-740), belnacasan (VX-765), VX-043198, emricasan, nivocasan (GS-9450), NCX-1000, and pharmaceutically acceptable forms thereof.

In certain embodiments, the invention provides a method of treating cancer by enhancing innate immune responses in a subject in need thereof comprising the steps of: (1) administering to the subject a therapeutically effective amount of doxorubicin, and pharmaceutically acceptable forms thereof; and (2) administering to the subject a therapeutically effective amount of caspase inhibitor chosen from the group consisting of Cbz-VAD-FMK (ZVAD), Ac-YVAD-CHO, pralnacasan (VX-740), belnacasan (VX-765), VX-043198, emricasan, nivocasan (GS-9450), NCX-1000, and pharmaceutically acceptable forms thereof.

In certain embodiments, the invention provides a method of treating cancer by enhancing innate immune responses in a subject in need thereof comprising the steps of: (1) administering to the subject a therapeutically effective amount of an apoptosis initiating agent chosen from the group consisting of paclitaxel, docetaxel, cabazitaxel, and protein-bound paclitaxel, and pharmaceutically acceptable forms thereof; and (2) administering to the subject a therapeutically effective amount of caspase inhibitor chosen from the group consisting of Cbz-VAD-FMK (ZVAD), Ac-YVAD-CHO, pralnacasan (VX-740), belnacasan (VX-765), VX-043198, emricasan, nivocasan (GS-9450), NCX-1000, and pharmaceutically acceptable forms thereof.

In certain embodiments, the invention provides a method of treating cancer by enhancing innate immune responses in a subject in need thereof comprising the steps of: (1) administering to the subject a therapeutically effective amount of docetaxel, and pharmaceutically acceptable forms thereof; and (2) administering to the subject a therapeutically effective amount of caspase inhibitor chosen from the group consisting of Cbz-VAD-FMK (ZVAD), Ac-YVAD-CHO, pralnacasan (VX-740), belnacasan (VX-765), VX-043198, emricasan, nivocasan (GS-9450), NCX-1000, and pharmaceutically acceptable forms thereof.

In certain embodiments, the invention provides a method of treating cancer by enhancing innate immune responses in a subject in need thereof comprising the steps of: (1) administering to the subject a therapeutically effective amount of docetaxel, and pharmaceutically acceptable forms thereof; and (2) administering to the subject a therapeutically effective amount of emricasan, and pharmaceutically acceptable forms thereof.

In certain embodiments, the invention provides a method of treating cancer by promoting DNA sensing in cancer cells in a subject in need thereof comprising the steps of: (1) administering to the subject a therapeutically effective amount of an apoptosis initiating agent chosen from the group consisting of 3-hydroxy-3-methyl-glutaryl-CoA (HMGCoA) reductase inhibitors, and pharmaceutically acceptable forms thereof; and (2) administering to the subject a therapeutically effective amount of caspase inhibitor chosen from the group consisting of Cbz-VAD-FMK (ZVAD), Ac-YVAD-CHO, pralnacasan (VX-740), belnacasan (VX-765), VX-043198, emricasan, nivocasan (GS-9450), NCX-1000, and pharmaceutically acceptable forms thereof.

In certain embodiments, the invention provides a method of treating cancer by promoting DNA sensing in cancer cells in a subject in need thereof comprising the steps of: (1) administering to the subject a therapeutically effective amount of simvastatin, and pharmaceutically acceptable forms thereof; and (2) administering to the subject a therapeutically effective amount of emricasan, and pharmaceutically acceptable forms thereof.

In one specific embodiment, the invention provides a method of treating cancer by promoting DNA sensing in cancer cells in a subject in need thereof comprising the steps of: (1) administering to the subject a therapeutically effective amount of an apoptosis initiating agent chosen from the group consisting of doxorubicin, daunorubicin, epirubicin, idarubicin, mitoxantrone, valrubicin, and pharmaceutically acceptable forms thereof; and (2) administering to the subject a therapeutically effective amount of caspase inhibitor chosen from the group consisting of Cbz-VAD-FMK (ZVAD), Ac-YVAD-CHO, pralnacasan (VX-740), belnacasan (VX-765), VX-043198, emricasan, nivocasan (GS-9450), NCX-1000, and pharmaceutically acceptable form thereof.

In certain embodiments, the invention provides a method of treating cancer by promoting DNA sensing in cancer cells in a subject in need thereof comprising the steps of: (1) administering to the subject a therapeutically effective amount of doxorubicin, and pharmaceutically acceptable forms thereof; and (2) administering to the subject a therapeutically effective amount of caspase inhibitor chosen from the group consisting of Cbz-VAD-FMK (ZVAD), Ac-YVAD-CHO, pralnacasan (VX-740), belnacasan (VX-765), VX-043198, emricasan, nivocasan (GS-9450), NCX-1000, and pharmaceutically acceptable forms thereof.

In certain embodiments, the invention provides a method of treating cancer by promoting DNA sensing in cancer cells in a subject in need thereof comprising the steps of: (1) administering to the subject a therapeutically effective amount of doxorubicin, and pharmaceutically acceptable form thereof; and (2) administering to the subject a therapeutically effective amount of emricasan, and pharmaceutically acceptable form thereof.

In certain embodiments, the invention provides a method of treating cancer by promoting DNA sensing in cancer cells in a subject in need thereof comprising the steps of: (1) administering to the subject a therapeutically effective amount of an apoptosis initiating agent chosen from the group consisting of microtubule stabilizing agents, and pharmaceutically acceptable forms thereof; and (2) administering to the subject a therapeutically effective amount of caspase inhibitor chosen from the group consisting of Cbz-VAD-FMK (ZVAD), Ac-YVAD-CHO, pralnacasan (VX-740), belnacasan (VX-765), VX-043198, emricasan, nivocasan (GS-9450), NCX-1000, and pharmaceutically acceptable forms thereof.

In certain embodiments, the invention provides a method of treating cancer by promoting DNA sensing in cancer cells in a subject in need thereof comprising the steps of: (1) administering to the subject a therapeutically effective amount of an apoptosis initiating agent chosen from the group consisting of paclitaxel, docetaxel, cabazitaxel, protein-bound paclitaxel, and pharmaceutically acceptable forms thereof; and (2) administering to the subject a therapeutically effective amount of caspase inhibitor chosen from the group consisting of Cbz-VAD-FMK (ZVAD), Ac-YVAD-CHO, pralnacasan (VX-740), belnacasan (VX-765), VX-043198, emricasan, nivocasan (GS-9450), NCX-1000, and pharmaceutically acceptable forms thereof.

In certain embodiments, the invention provides a method of treating cancer by promoting DNA sensing in cancer cells in a subject in need thereof comprising the steps of: (1) administering to the subject a therapeutically effective amount of docetaxel, and pharmaceutically acceptable forms thereof; and (2) administering to the subject a therapeutically effective amount of caspase inhibitor chosen from the group consisting of Cbz-VAD-FMK (ZVAD), Ac-YVAD-CHO, pralnacasan (VX-740), belnacasan (VX-765), VX-043198, emricasan, nivocasan (GS-9450), NCX-1000, and pharmaceutically acceptable forms thereof.

In certain embodiments, the invention provides a method of treating cancer by promoting DNA sensing in cancer cells in a subject in need thereof comprising the steps of: (1) administering to the subject a therapeutically effective amount of docetaxel, and pharmaceutically acceptable forms thereof; and (2) administering to the subject a therapeutically effective amount of emricasan, and pharmaceutically acceptable forms thereof.

The combination therapy may also comprise one or more additional therapeutic agents. Radiation therapy can be enhanced using radiation modifiers and protectors. In one embodiment, a radiation modifier chosen from the group comprising of nicotinamide, etanidazole, misonidazole, nimorazole, mitomycin-C, tirapazamine, motexafin gadolinium, hafnium oxide nanoparticle (e.g. PEP503 or NBTXR3), local anesthetic (e.g. procaine and lidocaine), tranquilizers (e.g. chlorpromazine), trans sodium crocetinate, hyperbaric oxygen (e.g. NVX-108), carbogen (a mixture of 95% oxygen and 5% carbon dioxide), amifostine (WR-2721), irinotecan, taxanes, hyperthermia, N-ethylmaleimide, diamide, diethylmaleate, fludarabine, gemcitabine, hydroxyurea, bromodeoxyuridine, iododeoxyuridine, 5-Fluorouracil, and Fluorodeoxyuridine.

The additional therapeutic agent may be, e.g., a chemotherapeutic, a biotherapeutic agent (including but not limited to antibodies to VEGF, VEGFR, EGFR, Her2/neu, other growth factor receptors, CD20, CD40, CD-40L, CTLA-4, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNa2, GM-CSF), and cells transfected with genes encoding immune stimulating cytokines such as but not limited to GM-CSF). The additional active agent(s) may be administered in a single dosage form with one or more co-administered agent selected from the agent that initiates apoptosis, caspase inhibitor, the PD-1 antagonist; or the additional active agent(s) may be administered in separate dosage form(s) from the dosage forms containing the agent that initiates apoptosis, caspase inhibitor, and/or the PD-1 antagonist.

The therapeutic combination disclosed herein may be used in combination with one or more other active agents, including but not limited to, other anti-cancer agents that are used in the prevention, treatment, control, amelioration, or reduction of risk of a particular disease or condition (e.g., cell-proliferation disorders). In one embodiment, a compound disclosed herein is combined with one or more other anti-cancer agents for use in the prevention, treatment, control amelioration, or reduction of risk of a particular disease or condition for which the compounds disclosed herein are useful. Such other active agents may be administered, by a route and in an amount commonly used therefor, contemporaneously or sequentially with a compound of the present disclosure.

The additional active agent(s) may be one or more agents selected from the group consisting of anti-viral compounds, antigens, adjuvants, anti-cancer agents, STING (Stimulator of Interferon Genes) agonists, TLR (Toll-like receptor) agonists, CTLA-4, LAG-3 and PD-1 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including but not limited to anticancer vaccines. It will be understood the descriptions of the above additional active agents may be overlapping. It will also be understood that the treatment combinations are subject to optimization, and it is understood that the best combination to use of the agent that initiates apoptosis, the caspase inhibitor, the PD-1 antagonist, and one or more additional active agents will be determined based on the individual patient needs.

When the therapeutic combination disclosed herein is used contemporaneously with one or more other active agents, the agent that initiates apoptosis, the caspase inhibitor, the PD-1 antagonist may be administered either simultaneously with, or before or after, one or more other active agent(s). Any of the agent that initiates apoptosis, the caspase inhibitor, the PD-1 antagonist may be administered separately, by the same or different route of administration, or together in the same pharmaceutical composition as the other agent(s).

The weight ratio of the agent that initiates apoptosis, caspase inhibitor, the PD-1 antagonist may be varied and will depend upon the therapeutically effective dose of each agent. Generally, a therapeutically effective dose of each will be used. Combinations including at least one the agent that initiates apoptosis, at least one caspase inhibitor, and/or at least one PD-1 antagonist, and other active agents will generally include a therapeutically effective dose of each active agent. In such combinations, the agent that initiates apoptosis, the caspase inhibitor, and/or the PD-1 antagonist disclosed herein and other active agents may be administered separately or in conjunction. In addition, the administration of one element may be prior to, concurrent with, or subsequent to the administration of other agent(s).

In one embodiment, the invention provides an agent that initiates apoptosis, a caspase inhibitor, and/or a PD-1 antagonist, and at least one other active agent as a combined preparation for simultaneous, separate or sequential use in therapy. In one embodiment, the therapy is the treatment of cancer.

In one embodiment, the disclosure provides a kit comprising two or more separate pharmaceutical compositions, one of which contains an agent that initiates apoptosis, another of which contains a caspase inhibitor, and/or another of which contains a PD-1 antagonist. In one embodiment, the kit comprises means for separately retaining said compositions, such as a container, divided bottle, or divided foil packet. A kit of the invention may be used for administration of different dosage forms, for example, oral and parenteral, for administration of the separate compositions at different dosage intervals, or for titration of the separate compositions against one another. To assist with compliance, a kit of the disclosure typically comprises directions for administration.

The invention also provides the use of a combination of an agent that initiates apoptosis and a caspase inhibitor for treating a cell-proliferation disorder, where the patient has previously (e.g., within 24 hours) been treated with a PD-1 antagonist. The disclosure also provides the use of a PD-1 antagonist for treating a cell-proliferation disorder, where the patient has previously (e.g., within 24 hours) been treated with a combination of an agent that initiates apoptosis and a caspase inhibitor.

Anti-viral compounds that may be used in combination with the therapeutic combinations disclosed herein include hepatitis B virus (HBV) inhibitors, hepatitis C virus (HCV) protease inhibitors, HCV polymerase inhibitors, HCV NS4A inhibitors, HCV NS5A inhibitors, HCV NS5b inhibitors, and human immunodeficiency virus (HIV) inhibitors.

Antigens and adjuvants that may be used in combination with the therapeutic combinations disclosed herein include B7 costimulatory molecule, interleukin-2, interferon-γ, GM-CSF, CTLA-4 antagonists, OX-40/0X-40 ligand, CD40/CD40 ligand, sargramostim, levamisol, vaccinia virus, Bacille Calmette-Guerin (BCG), liposomes, alum, Freund's complete or incomplete adjuvant, detoxified endotoxins, mineral oils, surface active substances such as lipolecithin, pluronic polyols, polyanions, peptides, and oil or hydrocarbon emulsions.

Adjuvants, such as aluminum hydroxide or aluminum phosphate, can be added to increase the ability of the vaccine to trigger, enhance, or prolong an immune response. Additional materials, such as cytokines, chemokines, and bacterial nucleic acid sequences, like CpG, a toll-like receptor (TLR) agonist as well as additional agonists for TLR-2, TLR-4, TLR-5, TLR-7, TLR-8, TLR-9, STING, including lipoprotein, lipopolysaccharide (LPS), monophosphoryllipid A, lipoteichoic acid, imiquimod, resiquimod, and in addition retinoic acid-inducible gene I (RIG-I) agonists such as poly I:C, used separately or in combination are also potential adjuvants.

Examples of cytotoxic agents that may be used in combination with the therapeutic combinations disclosed herein include, but are not limited to, arsenic trioxide, asparaginase (also known as L-asparaginase, and Erwinia L-asparaginase). Chemotherapeutic agents that may be used in combination with the therapeutic combinations disclosed herein include AEE788, abiraterone acetate, altretamine, AMG510, AMG706, aminopterin, anhydrovinblastine, anthracenedione, ARQ197, nelarabine, asparaginase, atorvastatin, auristatin, axitinib, azacitidine, AZD0530, AZD2171, AZD6244, belotecan, bendamustine, beta-lapachone, bevacizumab, BI2536, BIBF1120, bleomycin, BMS-275183, bortezomib, bosutinib, busulfan, camptothecin, capecitabine, carboplatin, carboquone, carmustine, CEP701, cetuximab, chlorambucil, chlormethine, CI-1033, cisplatin, cladribine, clofarabine, cyclophosphamide, cytarabine, dacarbazine, dactinomycin, dasatinib, docetaxel, EKB-569, EMD-72000, enocitabine, enzastaurin, epirubicin, erlotinib, ET-743, etoposide, everolimus, EXEL0999, EXEL7647, floxuridine, fludarabine phosphate, fotemustine, gefitinib, gemcitabine, GX15-070, HKI-272, hydroxyurea, ICR-62, idarubicin, ifosfamide, imatinib, irinotecan, ispinesib, ixabepilone, lapatinib, larotaxel, leucovorin, lomustine, lovastatin, mannosulfan, mechlorethamine, melphalan, mercaptopurine, methotrexate, mitomycin C, mitoxantrone, MLN-518, MS-275, napabucasin, nedaplatin, nilotinib, nimustine, ortataxel, osimertinib, oxaliplatin, paclitaxel, panitumumab, pazopanib, PD0325901, pemetrexed, pentostatin, PKC-412, plicamycin, prednimustine, procarbazine, PTK787, raltitrexed, ranimustine, rubitecan, sapacitabine, satraplatin, seliciclib, semustine, simvastatin, sorafenib, streptozocin, sunitinib, taxol, temozolomide, temsirolimus, teniposide, tesetaxel, thioguanine, thiotepa, topotecan, treosulfan, triaziquone, triethylenemelamine, triplatin tetranitrate, trofosfamide, uracil mustard, uramustine, vandetanib, vinblastine, vincristine, vindesine, vinorelbine, vinzolidine, XL119, XL880, and ZD6474.

The therapeutic agents and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art.

In one embodiment, the caspase inhibitor, e.g. emricasan is administered at a dose of about 0.5 mg, about 1 mg, about 5 mg, about 10 mg, about 20 mg, about 40 mg, about 80 mg, about 150 mg, about 200 mg, about 250 mg. Such doses may be provided, e.g., intratumorally, intravenously, subcutaneously, topically, orally, nasally, rectally, intramuscular, intracerebrally, intraspinally, or by inhalation. Such doses may be for once daily administration, twice daily administration, or every two days administration.

In another embodiment, the caspase inhibitor, e.g. emricasan is administered at a dose in a range of about 0.5 mg to about 250 mg, about 1 mg to about 100 mg, about 5 mg to about 50 mg, about 0.5 mg, about 5 mg, about 25 mg. Such doses may be provided, e.g., intratumorally, intravenously, subcutaneously, topically, orally, nasally, rectally, intramuscular, intracerebrally, intraspinally, or by inhalation. Such doses may be for once daily administration, twice daily administration, or every two days administration.

In another embodiment, the caspase inhibitor, e.g. emricasan, is administered at a dose of about 0.5 mg delivered intratumorally, about 5 mg delivered intratumorally, about 10 mg delivered intratumorally, about 20 mg delivered intratumorally, about 40 mg delivered intratumorally, about 80 mg delivered intratumorally, about 150 mg delivered intratumorally, about 200 mg delivered intratumorally, about 250 mg delivered intratumorally, e.g. about 250 mg/day. Such doses may be particularly adapted for patients of weight between 50 and 120 kg, e.g. 70 and 100 kg.

In some embodiments, the caspase inhibitor, e.g. emricasan, is administered at a dose in a range of about 0.5 mg/day, 1 mg/day, about 5 mg/day, about 10 mg/day, about 15 mg/day, about 20 mg/day, about 25 mg/day, about 30 mg/day, about 40 mg/day, about 50 mg/day, about 75 mg/day, about 100 mg/day delivered intratumorally. Such doses may be also provided, e.g., intratumorally, intravenously, subcutaneously, topically, orally, nasally, rectally, intramuscular, intracerebrally, intraspinally, or by inhalation. Such regimens may be particularly adapted for patients of weight between 50 and 120 kg, e.g. 70 and 100 kg.

In some embodiments of the invention, the caspase inhibitor, e.g. emricasan, is administered intratumorally at a dose of about 0.5 mg twice daily, about 5 mg twice daily, about 10 mg twice daily, about 25 mg twice daily, about 50 mg twice daily, about 75 mg twice daily, about 100 mg twice daily. Such doses may be also provided, e.g., intratumorally, intravenously, subcutaneously, topically, orally, nasally, rectally, intramuscular, intracerebrally, intraspinally, or by inhalation.

In one embodiment, the pharmaceutical combination, e.g fixed or free combination, comprises (1) about 0.1 mg to about 50 mg docetaxel; and (2) about 0.5 mg to about 50 mg of emricasan. For example, the pharmaceutical combination, e.g fixed or free combination, comprises (1) about 0.1 mg, about 5 mg, about 10 mg, about 20 mg, about 30 mg, about 40 mg, about 50 mg, about 100 mg of docetaxel, in free form or as a pharmaceutically acceptable salt thereof; and (2) about 0.5 mg or 5 mg or 10 mg or 25 mg or 50 mg or 100 mg or 200 mg or 250 mg of emricasan, in free form or as a pharmaceutically acceptable salt thereof. Such combinations can be delivered intratumorally.

PD-1 antagonists may be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total dose for a treatment interval is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/kg, 10 mg/kg, 25 mg/kg, 50 mg/kg or more. Doses may also be provided to achieve a pre-determined target concentration of PD-1 antagonists in the subject's serum, such as 0.1, 0.3, 1, 3, 10, 30, 100, 300 μg/mL or more. In one embodiment, the PD-1 antagonist is administered as a 200 mg dose once every 21 days. In other embodiments, PD-1 antagonists are administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

In one embodiment, the subject in need thereof is administered a biologically equivalent dose (BED) of radiation therapy. In another embodiment, the subject in need thereof is administered time-adjusted BED. In another embodiment, the subject in need thereof is administered a hyperfractionation (reduced fraction size, two times or more per day) dose of radiation therapy. In another embodiment, the subject in need thereof is administered a hypofractionation (fewer fractions, larger dose-per-fraction) dose of radiation therapy.

In one embodiment, the radiation therapy is administered in a total dose of about 10 Gy to about 200 Gy. In another embodiment, the radiation therapy is administered at a total dose of about 10 Gy, about 20 Gy, about 30 Gy, about 40 Gy, about 50 Gy, about 60 Gy, about 70 Gy, about 80 Gy, about 90 Gy, about 100 Gy, about 110 Gy, about 120 Gy, about 130 Gy, about 140 Gy, about 150 Gy, about 160 Gy, about 170 Gy, about 180 Gy, about 190 Gy, about 200 Gy. Such doses may be administered three times daily, twice daily, once daily, 1-7 times per week, weekly, bi-weekly, monthly, bimonthly.

In one embodiment, the pharmaceutical combination, e.g fixed or free combination, comprises (1) a total dose of about 10 Gy to about 200 Gy radiation therapy; (2) about 0.5 mg to about 250 mg of emricasan; and (3) about 0.05 μg/kg body weight to about 50 mg/kg body weight of PD-1 pathway antagonist. For example, the pharmaceutical combination, e.g fixed or free combination, comprises (1) a total dose of about 10 Gy, about 20 Gy, about 30 Gy, about 40 Gy, about 50 Gy, about 60 Gy, about 70 Gy, about 80 Gy, about 90 Gy, about 100 Gy, about 110 Gy, about 120 Gy, about 130 Gy, about 140 Gy, about 150 Gy, about 160 Gy, about 170 Gy, about 180 Gy, about 190 Gy, about 200 Gy of radiation therapy; (2) about 0.5 mg or 5 mg or 10 mg or 20 mg or 50 mg or 100 mg of emricasan, in free form or as a pharmaceutically acceptable salt thereof; and (3) about 0.05 μg/kg body weight, about 0.2 μg/kg, about 0.5 μg/kg, about 1 μg/kg, about 10 μg/kg, about 100 μg/kg, about 0.25 mg/kg, about 1.0 mg/kg, about 2.0 mg/kg, about 5.0 mg/kg, about 10 mg/kg, about 25 mg/kg, about 50 mg/kg of PD-1 pathway antagonist, in free form or as a pharmaceutically acceptable salt thereof.

In one embodiment, the pharmaceutical combination, e.g fixed or free combination, comprises (1) about 0.1 mg to about 100 mg docetaxel; (2) about 0.5 mg to about 100 mg of emricasan; and (3) about 0.05 μg/kg body weight to about 50 mg/kg body weight of PD-1 pathway antagonist. For example, the pharmaceutical combination, e.g fixed or free combination, comprises (1) about 0.1 mg, about 5 mg, about 10 mg, about 20 mg, about 30 mg, about 40 mg, about 50 mg, about 100 mg of docetaxel, in free form or as a pharmaceutically acceptable salt thereof; (2) about 0.5 mg or 5 mg or 10 mg or 25 mg or 50 mg or 100 mg of emricasan, in free form or as a pharmaceutically acceptable salt thereof; and (3) about 0.05 μg/kg body weight, about 0.2 μg/kg, about 0.5 μg/kg, about 1 μg/kg, about 10 μg/kg, about 100 μg/kg, about 0.25 mg/kg, about 1.0 mg/kg, about 2.0 mg/kg, about 5.0 mg/kg, about 10 mg/kg, about 25 mg/kg, about 50 mg/kg of PD-1 pathway antagonist, in free form or as a pharmaceutically acceptable salt thereof.

Various types of cancer may be treated with compositions and methods disclosed herein. The terms “cancer”, “cancerous”, or “malignant”, as used herein, refer to or describe the physiological condition in mammals that is typically characterized by unregulated cell growth.

In certain embodiments, the cancer that may be treated with compositions and methods disclosed herein is selected from lung cancer, prostate cancer, pancreatic cancer, ovarian cancer, cervical cancer, esophageal cancer, gastroesophageal junction cancer, gastric cancer, colon cancer, colorectal cancer, head and neck cancer, brain cancer, gliomas, astrocytomas, glioblastoma multiforme, Bannayan-Zonana syndrome, Cowden disease, Lhermitte-Duclos disease, inflammatory breast cancer, Wilm's tumor, Ewing's sarcoma, rhabdomyosarcoma, ependymoma, medulloblastoma, breast cancer, uterine cancer, kidney cancer, bladder cancer, liver cancer, vulvar cancer, cancer of the peritoneum, thyroid cancer, sarcomas, squamous cell cancer, melanoma, salivary gland cancer, hepatocellular cancer, leukemia, lymphoma, myeloma, GIST (gastrointestinal stromal), testicular cancer, and cancers of unknown primary (i.e., cancers in which a metastasized cancer is found but the original cancer site is not known). In particular embodiments, the cancer is present in an adult patient; in additional embodiments, the cancer is present in a pediatric patient. In particular embodiments, the cancer is AIDS-related.

In certain embodiments, the cancer is selected from brain and spinal cancers. In particular embodiments, the brain and spinal cancer is selected from the group consisting of anaplastic astrocytomas, glioblastomas, astrocytomas, and estheosioneuroblastomas (also known as olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astrocytoma/malignant glioma, visual pathway and hypothalamic glioma, and primary central nervous system lymphoma. In specific instances of these embodiments, the brain cancer is selected from the group consisting of glioma, glioblastoma multiforme, paraganglioma, and supratentorial primordial neuroectodermal tumors (sPET).

In certain embodiments, the cancer is selected from cancers of the head and neck, including recurrent or metastatic head and neck squamous cell carcinoma (HNSCC), nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers. In particular embodiments, the ocular cancer is selected from the group consisting of intraocular melanoma and retinoblastoma.

In certain embodiments, the cancer is selected from leukemia and cancers of the blood. In particular embodiments, the cancer is selected from the group consisting of myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic.

In certain embodiments, the cancer is selected from skin cancers. In particular embodiments, the skin cancer is selected from the group consisting of melanoma, squamous cell cancers, and basal cell cancers. In specific embodiments, the skin cancer is unresectable or metastatic melanoma.

In certain embodiments, the cancer is selected from cancers of the reproductive system. In particular embodiments, the cancer is selected from the group consisting of breast cancers, cervical cancers, vaginal cancers, ovarian cancers, endometrial cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In more specific instances of these embodiments, the breast cancer is triple-negative breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In certain embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gallbladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, and anal squamous cell carcinomas.

In certain embodiments, the cancer is selected from liver and bile duct cancers. In particular embodiments, the cancer is liver cancer (also known as hepatocellular carcinoma). In particular embodiments, the cancer is bile duct cancer (also known as cholangiocarcinoma); in instances of these embodiments, the bile duct cancer is selected from the group consisting of intrahepatic cholangiocarcinoma and extrahepatic cholangiocarcinoma.

In certain embodiments, the cancer is selected from kidney and bladder cancers. In particular embodiments, the cancer is a kidney cancer selected from the group consisting of renal cell cancer, Wilms tumors, and transitional cell cancers. In particular embodiments, the cancer is a bladder cancer selected from the group consisting of urothelial carcinoma (a transitional cell carcinoma), squamous cell carcinomas, and adenocarcinomas.

In certain embodiments, the cancer is selected from bone cancers. In particular embodiments, the bone cancer is selected from the group consisting of osteosarcoma, malignant fibrous histiocytoma of bone, Ewing sarcoma, chordoma (cancer of the bone along the spine).

In certain embodiments, the cancer is selected from lung cancers. In particular embodiments, the lung cancer is selected from the group consisting of non-small cell lung cancer, small cell lung cancers, bronchial tumors, and pleuropulmonary blastomas.

In certain embodiments, the cancer is selected from malignant mesothelioma. In particular embodiments, the cancer is selected from the group consisting of epithelial mesothelioma and sarcomatoid. In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In certain embodiments, the cancer is selected from lymphomas. In particular embodiments, the cancer is selected from the group consisting of Hodgkin lymphoma (e.g., Reed-Sternberg cells), non-Hodgkin lymphoma (e.g., diffuse large B-cell lymphoma, follicular lymphoma, mycosis fungoides, Sezary syndrome, primary central nervous system lymphoma), cutaneous T-cell lymphomas, primary central nervous system lymphomas.

In certain embodiments, the cancer is selected from glandular cancers. In particular embodiments, the cancer is selected from the group consisting of adrenocortical cancer (also known as adrenocortical carcinoma or adrenal cortical carcinoma), pheochromocytomas, paragangliomas, pituitary tumors, thymoma, and thymic carcinomas.

In certain embodiments, the cancer is selected from thyroid cancers. In particular embodiments, the thyroid cancer is selected from the group consisting of medullary thyroid carcinomas, papillary thyroid carcinomas, and follicular thyroid carcinomas.

In certain embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In certain embodiments, the cancer is selected from heart tumors. In particular embodiments, the heart tumor is selected from the group consisting of malignant teratoma, lymphoma, rhabdomyosarcoma, angiosarcoma, chondrosarcoma, infantile fibrosarcoma, and synovial sarcoma.

In certain embodiments, the cell-proliferation disorder is selected from benign papillomatosis, benign neoplastic diseases and gestational trophoblastic diseases. In particular embodiments, the benign neoplastic disease is selected from skin papilloma (warts) and genital papilloma. In particular embodiments, the gestational trophoblastic disease is selected from the group consisting of hydatidiform moles, and gestational trophoblastic neoplasia (e.g., invasive moles, choriocarcinomas, placental -site trophoblastic tumors, and epithelioid trophoblastic tumors). In embodiments, the cell-proliferation disorder is a cancer that has metastasized, for example, liver metastases from colorectal cancer.

In certain embodiments, the cell-proliferation disorder is selected from the group consisting of solid tumors and lymphomas. In particular embodiments, the cell-proliferation disorder is selected from the group consisting of advanced or metastatic solid tumors and lymphomas. In more particular embodiments, the cell-proliferation disorder is selected from the group consisting of malignant melanoma, head and neck squamous cell carcinoma, breast adenocarcinoma, and lymphomas. In aspects of such embodiments, the lymphomas are selected from the group consisting of diffuse large B-cell lymphoma, follicular lymphoma, mantle cell lymphoma, small lymphocytic lymphoma, mediastinal large B-cell lymphoma, splenic marginal zone B-cell lymphoma, extranodal marginal zone B-cell lymphoma of mucosa-associated lymphoid tissue (malt), nodal marginal zone B-cell lymphoma, lymphoplasmacytic lymphoma, primary effusion lymphoma, Burkitt lymphoma, anaplastic large cell lymphoma (primary cutaneous type), anaplastic large cell lymphoma (systemic type), peripheral T-cell lymphoma, angioimmunoblastic T-cell lymphoma, adult T-cell lymphoma/leukemia, nasal type extranodal KIT-cell lymphoma, enteropathy-associated T-cell lymphoma, gamma/delta hepatosplenic T-cell lymphoma, subcutaneous panniculitis-like T-cell lymphoma, mycosis fungoides, and Hodgkin lymphoma.

In certain embodiments, the cell-proliferation disorder is classified as stage III cancer or stage IV cancer. In instances of these embodiments, the cancer is not surgically resectable.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

Any appropriate route of administration can be employed, for example, parenteral, intravenous, subcutaneous, intramuscular, intraventricular, intracorporeal, intraperitoneal, rectal, or oral administration. Most suitable means of administration for a particular patient will depend on the nature and severity of the disease or condition being treated or the nature of the therapy being used and on the nature of the active compound.

Compositions for parenteral injection comprise pharmaceutically-acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions can also contain adjuvants such as preservative, wetting agents, emulsifying agents, and dispersing agents. Prevention of the action of microorganisms may be ensured by the inclusion of various antibacterial and antifungal agents, for example, paragen, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

The following examples are meant to be illustrative of the practice of the invention, and not limiting in any way.

### EXAMPLES

Exemplary methods and materials are described herein, although methods and materials similar or equivalent to those described herein can also be used in the practice or testing of the present disclosure. The materials, methods, and examples are illustrative only and not intended to be limiting.

Cell lines and drugs: MC38 and B16 cell lines were derived from C57BL6 murine colon adenocarcinoma and melanoma cells, respectively. The cells were maintained in Dulbecco's modified Eagle's medium (DMEM) supplemented with 10% heat-inactivated fetal bovine serum (FBS) and 100 U/mL penicillin/streptomycin. The CT26 cells were derived from murine colon adenocarcinoma cells and cultured in RPMI1640 with 10% FBS and 2 mM GlutaMAX. The 3-hydroxy-3-methyl-glutaryl-CoA (HMGCoA) reductase inhibitor simvastatin, caspase inhibitor emricasan, Bcl2 inhibitor venetoclax, DNA damaging agent doxorubicin, and microtubule-stabilizing agent docetaxel were purchased from Chemscene.

Apoptosis flow cytometry assay: FITC Annexin V Apoptosis Detection Kit with PI (BioLegend) was used for apoptosis detection. The MC38 cells were seeded in 24-well plate with 1×10\hat{ }5 cells/well and cultured overnight. The DMSO drug stocks were diluted in DMEM to make the working solution as the drug concentrations with Venetoclax 3 μM, Doxorubicin 1 μM, Docetaxel 10 μM, and Simvastatin 10 μM, respectively. In the drug combination treatment groups, 10 μM Emricasan were added in the diluted Venetoclax, Doxorubicin, Docetaxel and Simvastatin DMEM medium to 10 μM. After 24 hours treatment, cells were collected and dissociated as single cell suspensions for Annexin V/PI staining. In briefing, cells were washed twice with cold BioLegend's Cell Staining Buffer, then resuspended in 100 mL Annexin V Binding Buffer. 5 mL FITC Annex V and 10 mL Propidium Iodide (PI) Solution were added in each sample. The samples were gently vortexed and incubated for 15 minutes at room temperature in the dark. Wash the samples with Cell Staining Buffer one time and resuspend cells in 500 mL Cell Staining Buffer for flow cytometry analysis. The data were recorded on BD Accuri™ C6 plus flow Cytometer and analyzed with FlowJo software.

Interferon-beta (IFNβ) ELISA assay: The MC38 cell culture supernatants were collected 24 hours post-drug treatment (simvastatin 10 μM, venetoclax 3 μM, doxorubicin 1 μM, and docetaxel 10 μM, respectively) and stored at −80° C. for IFNβ ELISA measurement. The concentration of IFNβ was measured with VeriKine-HS Interferon Beta Serum ELISA Kit (PBL Assay Science) in accordance with the manufacturer's instruction. The data of absorbance was recorded at 450 nm by using BioTek synergy 4 plate reader and analyzed with GraphPad software.

Caspase-3 activity assay: Caspase-3 activity was measured by using Sigma Caspase 3 Assay Kit, Fluorimetric. In brief, MC38 cells were cultured in 96-well plates and treated with Docetaxel and Docetaxel/Emricasan combination, respectively. Dose-dependent effect of Docetaxel on caspase-3 activity was tested in MC38 cells; Docetaxel/Emricasan combination treatment was applied to reverse Docetaxel induced caspase-3 activity. After 24 h drug treatment, the cells were lysed in 25 μL 1× lysis buffer on ice for 20 min, then added 200 μL of 16.7 μM Ac-DEVD-AMC substrate solution to incubate at RT in the dark for 30 min. The fluorescent signals were read in BioTek Synergy 4 plate reader at excitation 360 nm and emission 460 nm.

PD-L1 expression flow cytometry assay: Cell surface PD-L1 expression was detected by flow cytometry assay. The cells were plated in 24-well plates and treated with 1 μM Docetaxel, 10 μM Emricasan, and the combination of the two drugs. Untreated cells were used as a control. After 24 h of treatment, the cells were dissociated and harvested for PD-L1 staining with BioLegend PE anti-mouse PD-L1 antibody (MIH7 clone). The PD-L1 expression was measured in BD Accuri C6 Plus flow cytometer, The mean density of PD-L1 was analyzed by FlowJo V10 software.

In vivo studies: Female C57BL6 and Balb/C mice of 9 weeks of age were obtained from the Jackson lab, ME. Passages were made twice weekly with a 1:2 split and cultured in DMEM:F12 supplemented with 10% FBS (HyClone, Ft. Collins, Colo.). For inoculation, approximately 5-8×10⁵ cells were suspended in 100 μL of PBS (Becton Dickinson Labware, Bedford, Mass.) and injected subcutaneously into the flanks of mice. Most mice developed palpable tumors within 5 days of inoculation. Tumor bearing mice are grouped in cohorts of 5-8 mice for dosing studies. Mice with tumor sizes of 50-100 mm³ are randomized between treatment groups. Baseline tumor volumes were established and dosing initiation began on day 0. Emricasan was administered as a salt intratumorally or interperitoneally for 3-4 days. If docetaxel was used, it was administered interperitoneally or intravenously in Tween80/PEG300 or Tween80/ethanol with PBS the day before emricasan was given. Emricasan and docetaxel were co-formulated in Tween80/PEG300 and co-administered intratumorally after diluting with PBS. Tumor volumes were measured twice a week using standard calipers and calculated as (length×width)/2, with the length and width defined as the long and short axes, respectively. Measurement of body weight was initiated on day 0 and repeated once a week.

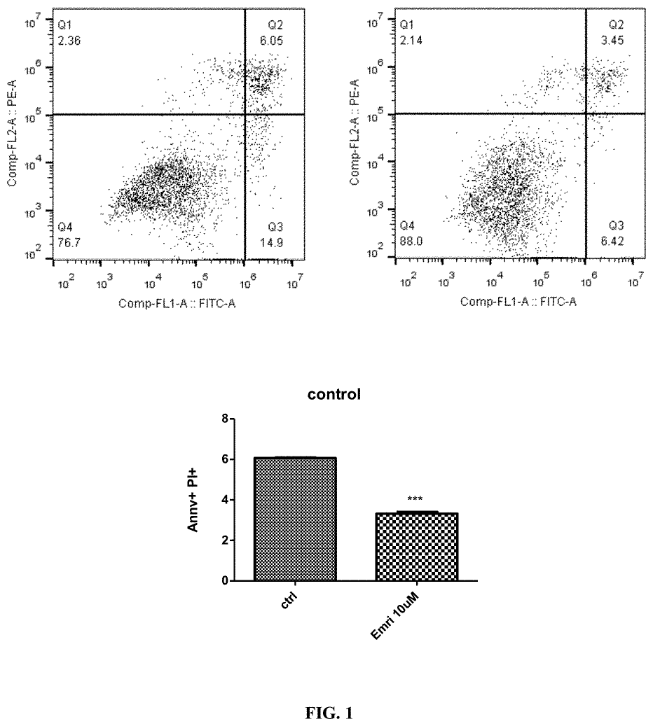

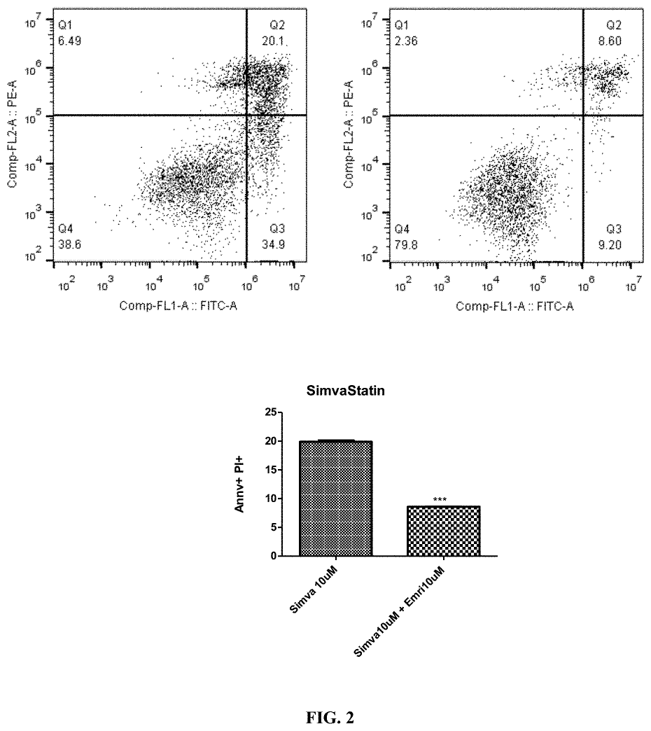

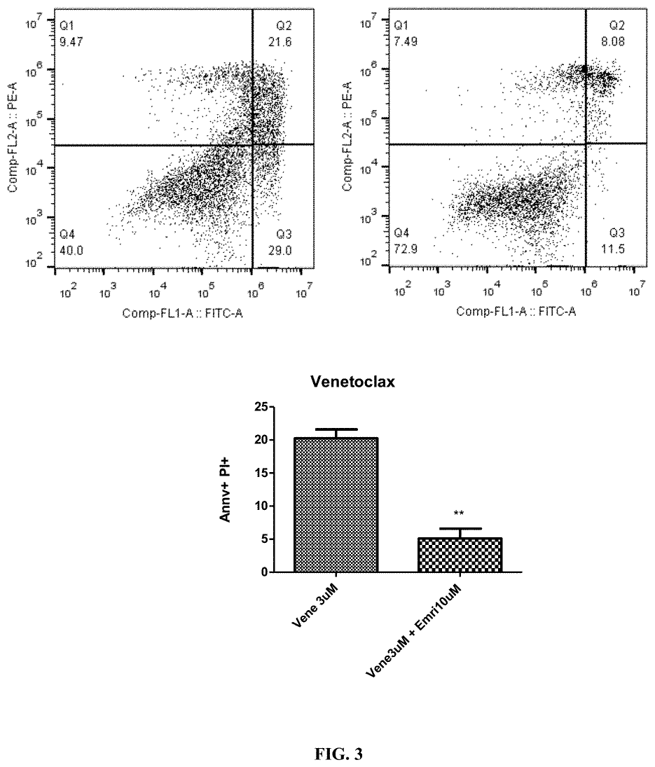

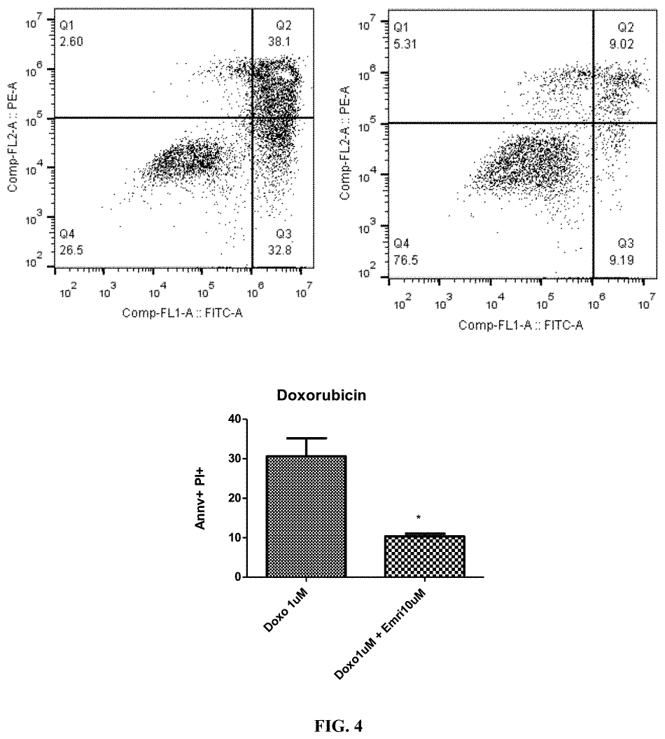

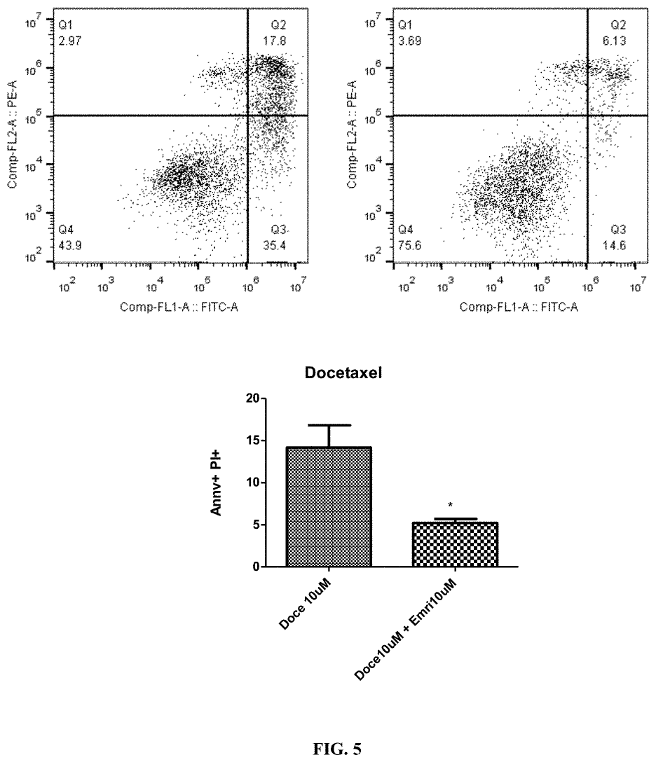

Our results demonstrated that caspase inhibitor emricasan partially blocked the apoptosis in MC38 cells as shown in FIG. 1. The 3-hydroxy-3-methyl-glutaryl-CoA (HMGCoA) reductase inhibitor simvastatin, Bcl2 inhibitor venetoclax, DNA damaging agent doxorubicin, and microtubule-stabilizing agent docetaxel all induced substantial apoptosis in MC38 cells. Addition of 10 μM of caspase inhibitor emricasan significantly reduced the apoptosis induced by these agents, as shown in FIG. 2, FIG. 3, FIG. 4, and FIG. 5, respectively.

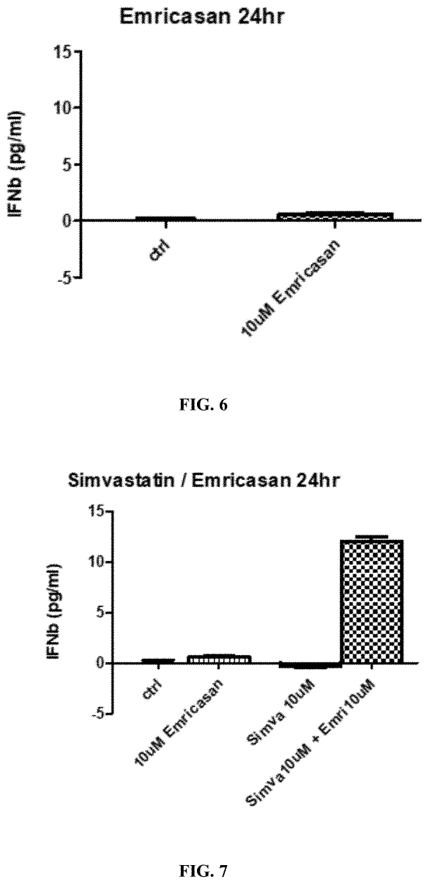

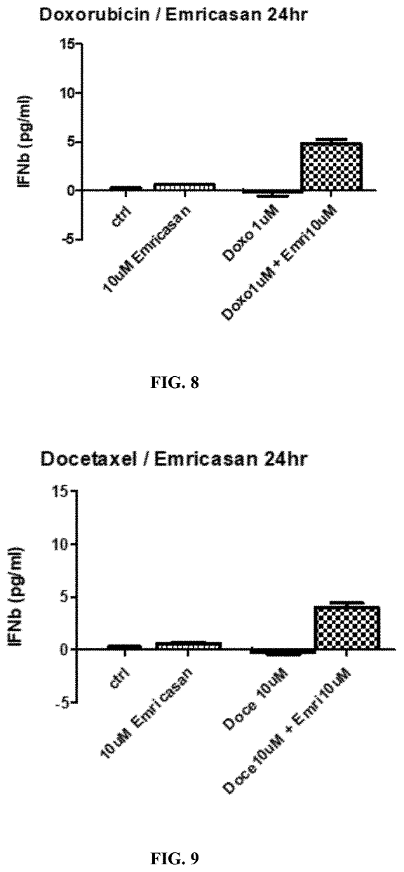

Our data demonstrated that caspase inhibitor emricasan slightly enhanced the IFNβ production in MC38 cells as shown in FIG. 6. Treatment of MC38 cells with the 3-hydroxy-3-methyl-glutaryl-CoA (HMGCoA) reductase inhibitor simvastatin, Bcl2 inhibitor venetoclax, DNA damaging agent doxorubicin, and microtubule-stabilizing agent docetaxel all slightly reduced IFNβ production. However, addition of 10 μM of caspase inhibitor emricasan significantly enhanced the IFNβ production in the 3-hydroxy-3-methyl-glutaryl-CoA (HMGCoA) reductase inhibitor simvastatin, DNA damaging agent doxorubicin, and microtubule-stabilizing agent docetaxel treated MC38 cells as shown in FIG. 7, FIG. 8, and FIG. 9, respectively.

Docetaxel showed cytotoxicity in several tumor cells and activated caspase-3 activity most efficiently at ˜1 μM. Emricasan began to effectively inhibit the caspase-3 activity at ˜1 μM but reached the maximum activity at ˜10 μM. The preferred ratio between docetaxel and emricasan lied between 1:20 to 1:2 where both the cytotoxicity of docetaxel and the IFN effect of emricasan peaked (FIG. 10a, FIG. 10b, FIG. 10c, FIG. 11a, FIG. 11b, and FIG. 11c).

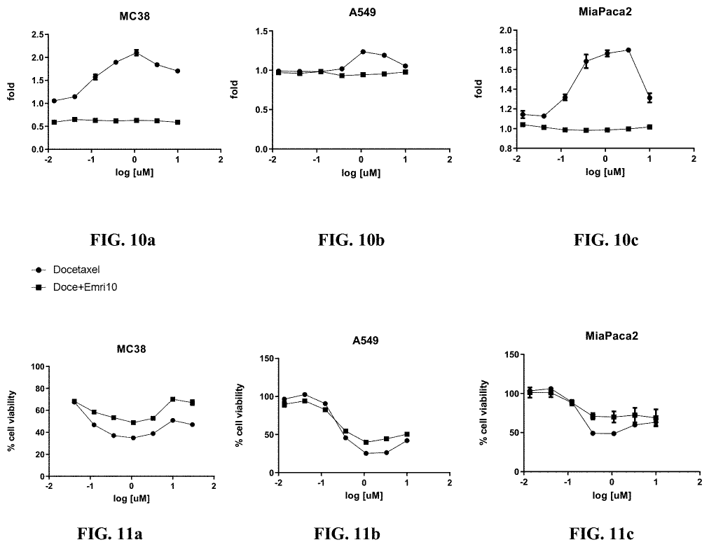

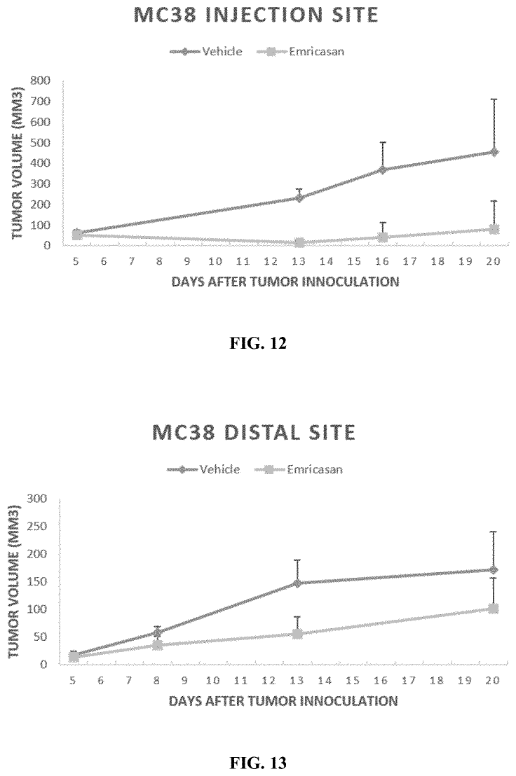

Intratumorally administered emricasan alone was able control small MC38 tumors (˜50 mm³) at the site of injection as shown in FIG. 12, but the distal tumor control was moderate as shown in FIG. 13.

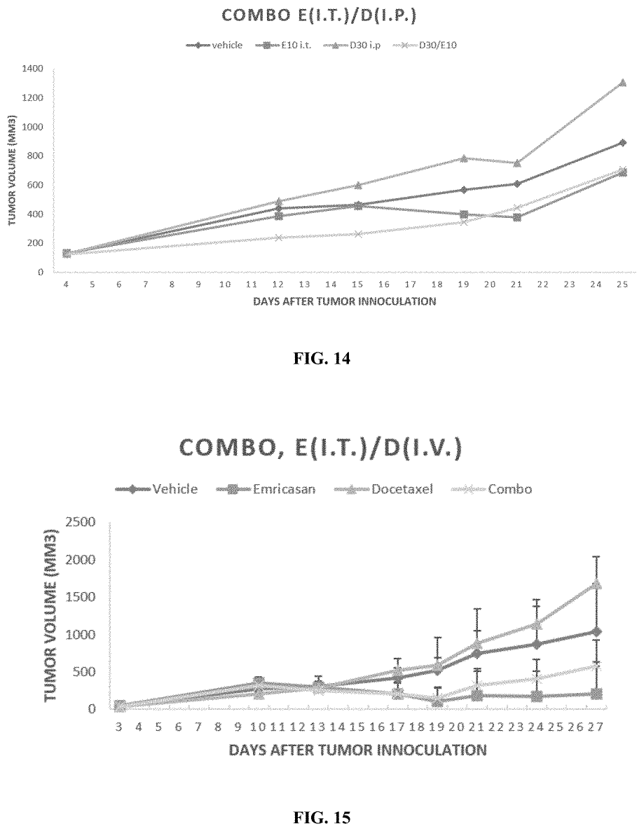

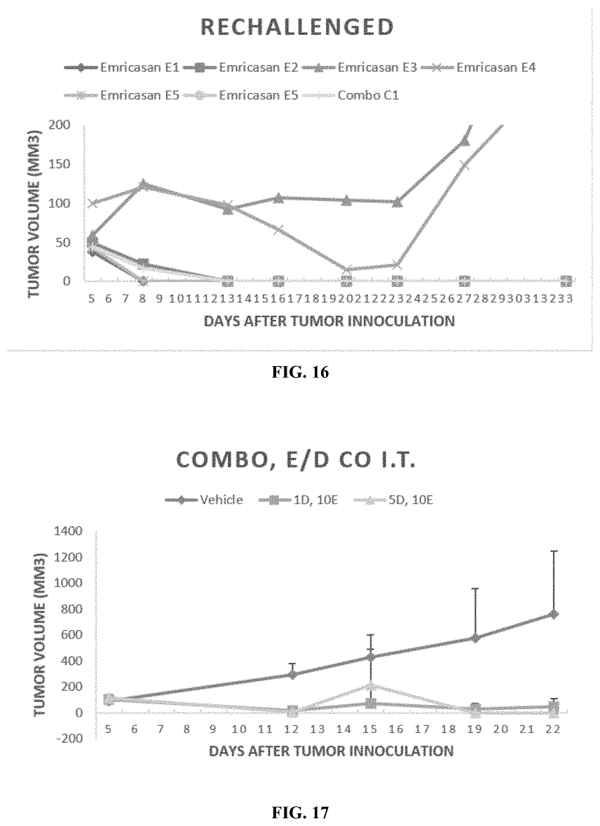

To control larger and later stage tumors (>100 mm³), the addition of docetaxel was necessary to achieve desirable efficacy. Systemically administered docetaxel as shown in FIG. 14 and FIG. 15 improved tumor control of emricasan, the maximum efficacy was achieved by co-administering docetaxel and emricasan intratumorally as shown in FIG. 17. Although intratumorally administered emricasan was unable to induce abscopal effect, the mice cured by emricasan and its combination with docetaxel were able to reject rechallenged tumor as shown in FIG. 16.

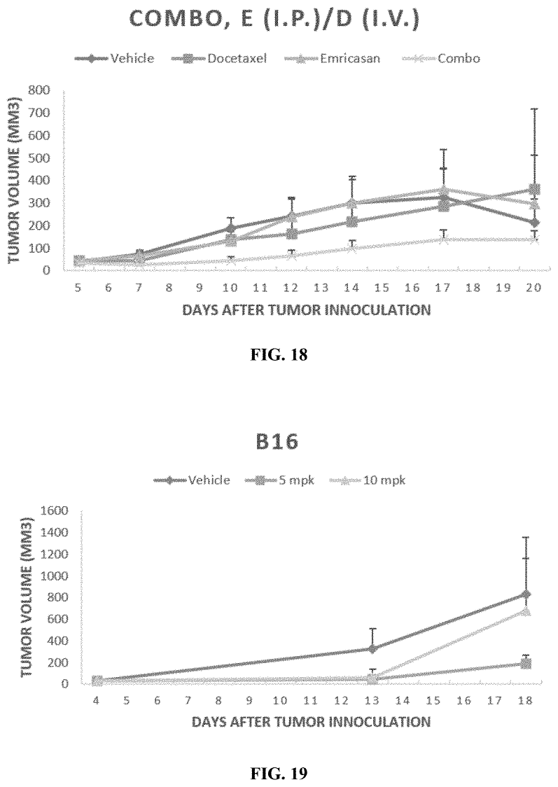

One method to control metastatic cancer was to administer docetaxel and emricasan systemically. As shown in FIG. 18, interperitoneally administered emricasan improved the systemic tumor control of intravenously administered docetaxel at high dose. Intratumorally administered emricasan and its combination with docetaxel upregulated the PD-L1 expression on tumor cells as shown in FIG. 21, which may have exacerbated systemic immune suppression. Another method to control metastatic cancer is to combine intratumorally administered emricasan, docetaxel or radiation therapy, and PD-1 pathway antagonists.

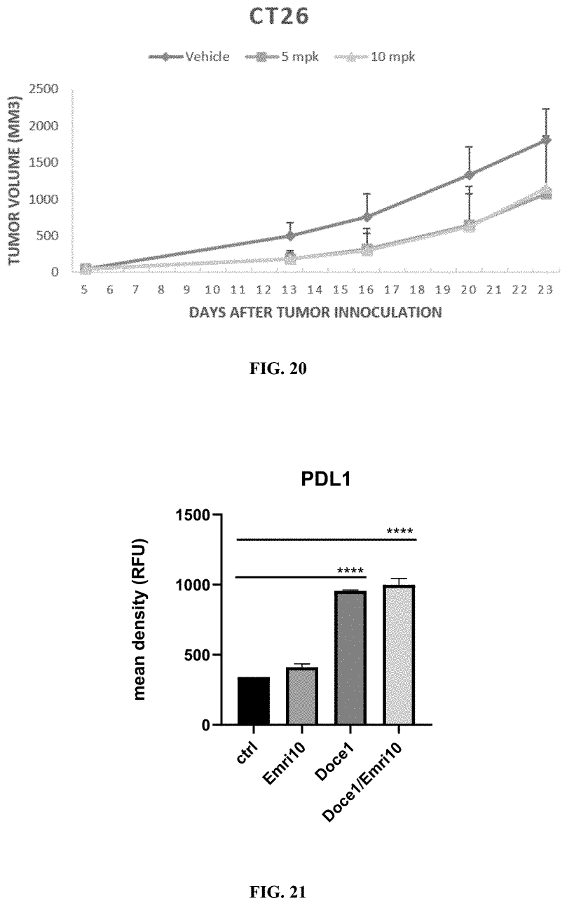

Intratumorally administered emricasan also produced tumor control efficacy in other tumor models as shown in FIG. 19 and FIG. 20.

IFNβ is a key early innate immune response signal that is responsible for several critical steps in anti-tumor immunity such as cross priming of CD8 T cells and is an integral component in STING and Toll-like receptor agonism as cancer therapeutics. The therapeutically induced apoptosis can be slowed down by caspase inhibition but cannot be reversed once apoptosis is committed. The induced IFNβ along with other cytokines such as TNFα can organize a more effective immune response to control tumor growth via the more inflammatory caspase-independent cell deaths in addition to the apoptosis initiated by radiation therapy or the chemotherapeutic agents.

Most cancers are diagnosed at the localized stage, but rationally designed anti-cancer drugs specifically targeting this large population are lacking. Most drugs were designed for metastatic diseases with the safety profiles incompatible with the treatment of early-stage cancer. Emricasan is a covalent inhibitor that is suitable for local administration as a short period of drug coverage would be sufficient to fully disable apoptotic caspases in the tumor tissue. The immunogenic nature of this mechanism also favors local administration since only tumor tissues and immune system are required for the IFN production.

Although intratumorally administered emricasan alone or in combination with intratumorally or systemically administered docetaxel produced moderate abscopal effect, the mice cured by emricasan were able to reject rechallenged tumors more efficiently. This indicates some level of immune memory was established and may prevent relapse after local tumor treatment. Intratumorally administered emricasan and docetaxel upregulate the PD-L1 expression, which may have exacerbated systemic immune suppression and comprised systemic efficacy. The addition of PD-1 pathway antagonists will control the local and systemic diseases more efficiently.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment. It is understood that wherever embodiments are described herein with the language “comprising,” otherwise analogous embodiments described in terms of “consisting of” and/or “consisting essentially of are also provided. Unless expressly stated to the contrary, all ranges cited herein are inclusive; i.e., the range includes the values for the upper and lower limits of the range as well as all values in between. As an example, dose ranges, percentages, and the like described herein include the upper and lower limits of the range and any value in the continuum there between.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A method for treating cancer, comprising intratumorally administering, simultaneously or sequentially, to a subject in need thereof a therapeutically effective amount of a caspase inhibitor and a therapeutically effective amount of an apoptosis inducer.

2. The method of claim 1, further comprising administering to the subject a therapeutically effective amount of a PD-1 pathway inhibitor.

3. The method of claim 1, wherein the caspase inhibitor is emricasan, or a pharmaceutically acceptable form thereof.

4. The method of claim 3, wherein emricasan is in the form of a base addition salt.

5. The method of claim 4, wherein the base addition salt is formed by emricasan and CH₃N(CH₂CH₂OH)₂ (N-methyl diethanolamine).

6. The method of claim 1, wherein the apoptosis inducer is docetaxel, or a pharmaceutically acceptable form thereof.

7. The method of claim 1, wherein the apoptosis inducer is radiation therapy.

8. The method of claim 2, wherein the PD-1 pathway inhibitor is selected from pembrolizumab, nivolumab, cemiplimab, spartalizumab, camrelizumab, sintilimab, tislelizumab, toripalimab, dostarlimab, INCMGA00012, AMP-224, AMP-514, atezolizumab, avelumab, durvalumab, and KN035.

9. The method of claim 1, wherein the cancer is localized.

10. The method of claim 1, wherein the cancer is metastatic.

11. The method of claim 1, wherein the cancer is selected from breast cancer, small cell lung cancer, non-small cell lung cancer, prostate cancer, gastric cancer, renal cell carcinoma, ovarian cancer, cervical cancer, colorectal cancer, hepatocellular carcinoma, glioblastoma, melanoma, basal cell carcinoma, cutaneous squamous cell carcinoma, Bowen's disease, pancreatic ductal carcinoma, head and neck squamous cell carcinoma, lip squamous cell carcinoma, buccal mucosa squamous cell carcinoma, oral tongue squamous cell carcinoma, oral squamous cell carcinoma, salivary mucoepidermoid carcinoma, and endometrial carcinoma.

12. (canceled)

13. A method for treating cancer, comprising intratumorally administering to a subject in need thereof a therapeutically effective amount of emricasan, or a pharmaceutically acceptable form thereof.

14. The method of claim 13, further comprising intratumorally administering to the subject a therapeutically effective amount of docetaxel, or a pharmaceutically acceptable form thereof.

15. The method of claim 14, wherein the therapeutically effective amount of emricasan and the therapeutically effective amount of docetaxel are co-administered as a single intratumoral administration.

16. The method of claim 14, wherein the therapeutically effective amount of emricasan and the therapeutically effective amount of docetaxel are co-administered as separate intratumoral administrations.

17. The method of claim 14, wherein the weight ratio of docetaxel and emricasan is in the range of about 1:20 to about 1:2.

18. (canceled)

19. The method of claim 14, wherein cancer growth at a site away from the site of intratumoral administration is suppressed.

20. The method of claim 14, wherein the intratumoral administration results in systemic suppression of cancer growth.

21. The method of claim 14, further comprising administering to the subject a PD-1 pathway inhibitor.

22-27. (canceled)

28. A method for treating cancer, comprising subcutaneously administering to a subject in need thereof a therapeutically effective amount of emricasan, or a pharmaceutically acceptable form thereof, and intravenously administering to the subject a therapeutically effective amount of docetaxel, or a pharmaceutically acceptable form thereof.

29-41. (canceled)
